(19) United States
(12) Patent Application Publication
(10) Pub. No.: US 2020/0254132 A1
(43) Pub. Date: Aug. 13, 2020

(54) STERILIZABLE LAMP ASSEMBLY OF VEHICLE AND STERILIZATION METHOD USING THE SAME

(71) Applicants: Hyundai Motor Company, Seoul (KR); Kia Motors Corporation, Seoul (KR)

(72) Inventor: Jae-Seung Lee, Hwaseong-Si (KR)

(73) Assignees: Hyundai Motor Company, Seoul (KR); Kia Motors Corporation, Seoul (KR)

(21) Appl. No.: 16/690,640

(22) Filed: Nov. 21, 2019

(30) Foreign Application Priority Data
Feb. 12, 2019 (KR) 10-2019-0016186

(51) Int. Cl. A61L 9/20; B60Q 3/80; B60Q 3/68; H05B 37/02

(52) U.S. Cl. CPC A61L 9/20; H05B 37/0227; B60Q 3/68; B60Q 3/80

(57) ABSTRACT

A sterilizable lamp assembly of a vehicle may include at least one sterilizing lamp mounted in an indoor of a vehicle and for irradiating sterilizable light of wavelength to the interior of the vehicle, wherein the sterilizing lamp is configured to emit light when a passenger does not get on the interior of the vehicle.

### CROSS-REFERENCE TO RELATED APPLICATION

The present application claims priority to Korean Patent Application No. 10-2019-0016186, filed on Feb. 12, 2019, the entire contents of which is incorporated herein for all purposes by this reference.

### BACKGROUND OF THE PRESENT INVENTION

### Field of the Invention

The present invention relates to a sterilizable lamp assembly for a vehicle and sterilization method using the same, which can selectively or simultaneously perform the indoor illumination and the sterilization function of the vehicle.

### Description of Related Art

The interior of the vehicle is provided with various illumination lamps to illuminate the inside when the surroundings are dark.

Furthermore, the interior of the vehicle is also provided with a mood lamp for mood illumination for inside interior, in addition to illumination for identifying objects.

Such an illumination lamp or a mood lamp simply irradiates light for indoor illumination or mood illumination when power is applied thereto.

Meanwhile, various equipments mounted in an indoor of a vehicle are easily exposed to bacteria that are transferred by a passenger's contact or received from the outside thereof through air or the like. For example, germs tend to breed in a door inside handle operated by the passenger when the passenger gets on or off, a steering wheel operated by a driver at operation, a seat occupied by the passenger, etc.

For the present purpose, conventionally, bacteria are removed by spraying or applying a germicide, etc. separately. However, sterilization by the germicide is time-consuming and thereby troublesome. Furthermore, since an effect is extinguished over time, there is a problem in that it is required to repeatedly perform a sterilization operation.

In recent years, vehicle sharing service is increasing, and in such a vehicle sharing service, the number of passengers using one vehicle is increased, increasing the pollution degree of the interior of the vehicle by bacteria. However, since the maintenance of the internal cleanliness of the vehicle is not so much controlled, the user of the vehicle sharing service is easily exposed to the bacteria-contaminated environment.

The information included in this Background of the present invention section is only for enhancement of understanding of the general background of the present invention and may not be taken as an acknowledgement or any form of suggestion that this information forms the prior art already known to a person skilled in the art.

### BRIEF SUMMARY

Various aspects of the present invention are directed to providing a sterilizable lamp assembly of a vehicle and sterilization method using the same, which can install a sterilizing lamp for irradiating sterilizable light of wavelength to an indoor of a vehicle to sterilize the interior of the vehicle by illumination the sterilizing lamp while a passenger does not get on and can illuminate an illumination lamp or a mood lamp when the passenger gets on.

A sterilizable lamp assembly of a vehicle according to an exemplary embodiment of the present invention for achieving the object includes at least one sterilizing lamp mounted in an indoor of a vehicle and for irradiating sterilizable light of wavelength to the interior of the vehicle, and the sterilizing lamp is configured to emit light when a passenger does not get on the interior of the vehicle.

The sterilizing lamp irradiates ultraviolet rays of 200 nm to 280 nm.

The sterilizing lamp is a UV-C LED.

An opening through which the light irradiated from the sterilizing lamp can pass is formed at an interior material of the vehicle, and the inside of the interior material includes a diffusion panel for dispersing the light irradiated from the sterilizing lamp.

The diffusion panel has a reflective surface, which has been formed to be inclined to reflect the light irradiated from the sterilizing lamp to the opening, repeatedly formed thereon.

The rear surface of the reflective surface is formed with a reflective layer on which chrome has been coated.

The sterilizable lamp assembly of the vehicle further includes a controller for turning on the sterilizing lamp, and an ICU for outputting a turning-on signal of the sterilizing lamp to the controller, when it is detected that the passenger has not gotten on the vehicle from a passenger detecting sensor for detecting whether the passenger has gotten on the interior of the vehicle.

The sterilizing lamp is mounted inside a handle housing of a door inside handle to illuminate the door inside handle.

The sterilizing lamp is mounted inside an air blowing duct configured for blowing air to the interior of the vehicle.

The sterilizing lamp is mounted at a crash pad of the vehicle to illuminate a mobile phone holder in which a mobile phone is configured to be held for wireless charging.

The sterilizing lamp is mounted at a headlining of the interior of the vehicle to illuminate the interior of the vehicle.

A mood lamp for mood-illuminating the interior of the vehicle is mounted at a position adjacent to the sterilizing lamp.

A sterilization method using a sterilizable lamp assembly of a vehicle includes in the sterilization method for sterilizing the interior of the vehicle by use of at least one sterilizing lamp mounted in the interior of the vehicle and for irradiating sterilizable light of wavelength to the interior of the vehicle, determining whether a passenger has gotten on the interior of the vehicle, and sterilizing the interior of the vehicle by turning on the sterilizing lamp, when it is determined that the passenger has not gotten on.

The sterilization method using the sterilizable lamp assembly of the vehicle further includes inputting a sensor value that inputs a sensor value from a passenger detecting sensor mounted in the vehicle to an ICU, before the determining whether the passenger has gotten on the vehicle.

The sterilizing includes starting sterilization illumination that outputs a turning-on signal of the sterilizing lamp to a controller, when it is determined that the passenger has not gotten on the vehicle by the ICU, and turning on the sterilizing lamp by applying power to the sterilizing lamp, when receiving the turning-on signal of the sterilizing lamp from the ICU by the controller.

The sterilization method using the sterilizable lamp assembly of the vehicle further include, after the sterilizing has been performed, determining whether to exceed a sterilization time that determines whether the time in which the sterilizing lamp has been turned on has exceeded a predetermined sterilization time for turning off the sterilizing lamp, and turning off the sterilizing lamp that turns off the sterilizing lamp, when it is determined that the time in which the sterilizing lamp has been turned on has exceeded the predetermined sterilization time.

In the determining whether to exceed the predetermined sterilization time, when it is determined that the time in which the sterilizing lamp has been turned on has not exceeded the predetermined sterilization time, the determining whether to exceed the predetermined sterilization time is repeatedly performed to maintain a state where the sterilizing lamp has been turned on.

In the determining whether the passenger has gotten on the vehicle, when it is determined that the passenger has gotten on the vehicle, the illuminating mood that mood-illuminates the interior of the vehicle with a mood lamp mounted adjacent to the sterilizing lamp is performed.

The illuminating mood includes starting the mood illumination that outputs a turning-on signal of the mood lamp to a controller when it is determined that the passenger has gotten on the vehicle by the ICU, and turning on the mood lamp that turns on the mood lamp by applying power to the mood lamp, when receiving the turning-on signal of the mood lamp from the ICU by the controller.

According to the sterilizable lamp assembly of the vehicle of the present invention having the above configuration and the sterilization method using the same, it is possible to sterilize the interior of the vehicle by illumination the sterilizing lamp in a state where the passenger does not get on by interlocking with whether the passenger has gotten on the vehicle, such that the passenger may be prevented from the bacterial infection.

It is possible to sterilize the interior of the vehicle even without performing a separate sterilization operation, facilitating the internal sterilization of the vehicle.

Furthermore, it is possible to sterilize only by illumination the sterilizing lamp even when a plurality of passengers use a single vehicle commonly, such as the vehicle sharing service, enhancing the cleanliness of the shared vehicle.

The methods and apparatuses of the present invention have other features and advantages which will be apparent from or are set forth in more detail in the accompanying drawings, which are incorporated herein, and the following Detailed Description, which together serve to explain certain principles of the present invention.

### BRIEF DESCRIPTION OF THE DRAWINGS

FIG. 1 is a schematic diagram illustrating an example to which a sterilizable lamp assembly of a vehicle according to an exemplary embodiment of the present invention is applied to a door inside handle.

FIG. 2 is a cross-sectional diagram illustrating an example to which the sterilizable lamp assembly of the vehicle according to an exemplary embodiment of the present invention is applied to the door inside handle.

FIG. 3 is an enlarged diagram of a portion A in FIG. 2.

FIG. 4 is a cross-section diagram illustrating a state where the sterilizable lamp assembly of the vehicle according to an exemplary embodiment of the present invention has been mounted in an air blowing duct of the vehicle.

FIG. 5 is a perspective diagram illustrating a state where the sterilizable lamp assembly of the vehicle according to various exemplary embodiments of the present invention has been applied to a mobile phone holder having the wireless charging function of the vehicle.

FIG. 6 is a perspective diagram illustrating a state where the sterilizable lamp assembly of the vehicle according to an exemplary embodiment of the present invention has been mounted at a headlining of the vehicle.

FIG. 7 is a flowchart illustrating a sterilization method using the sterilizable lamp assembly of the vehicle according to an exemplary embodiment of the present invention.

It may be understood that the appended drawings are not necessarily to scale, presenting a somewhat simplified representation of various features illustrative of the basic principles of the present invention. The specific design features of the present invention as included herein, including, for example, specific dimensions, orientations, locations, and shapes will be determined in part by the particularly intended application and use environment.

In the figures, reference numbers refer to the same or equivalent portions of the present invention throughout the several figures of the drawing.

### DETAILED DESCRIPTION

Reference will now be made in detail to various embodiments of the present invention(s), examples of which are illustrated in the accompanying drawings and described below. While the present invention(s) will be described in conjunction with exemplary embodiments of the present invention, it will be understood that the present description is not intended to limit the present invention(s) to those exemplary embodiments. On the other hand, the present invention(s) is/are intended to cover not only the exemplary embodiments of the present invention, but also various alternatives, modifications, equivalents and other embodiments, which may be included within the spirit and scope of the present invention as defined by the appended claims.

Hereinafter, a sterilizable lamp assembly for a vehicle according to an exemplary embodiment of the present invention and a sterilization method using the same will be described in detail with reference to the accompanying drawings.

A sterilizable lamp assembly of the vehicle according to an exemplary embodiment of the present invention includes at least one sterilizing lamp 22 mounted in an indoor of a vehicle and for irradiating sterilizable light of wavelength to the interior of the vehicle, and the sterilizing lamp 22 emits light when a passenger does not get on the vehicle.

The sterilizing lamp 22 is mounted in the indoor material of the vehicle to illuminate the interior of the vehicle for sterilization.

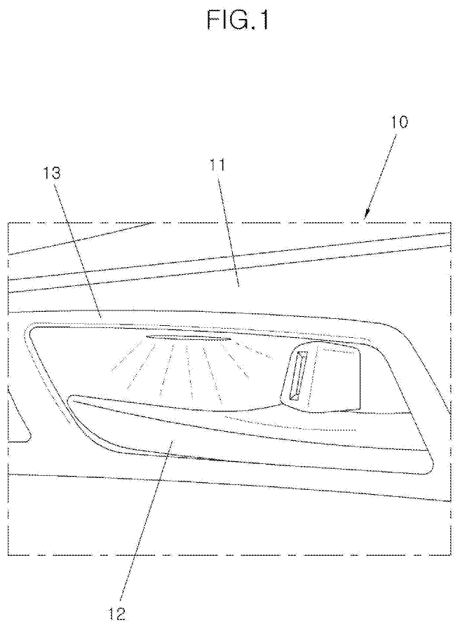

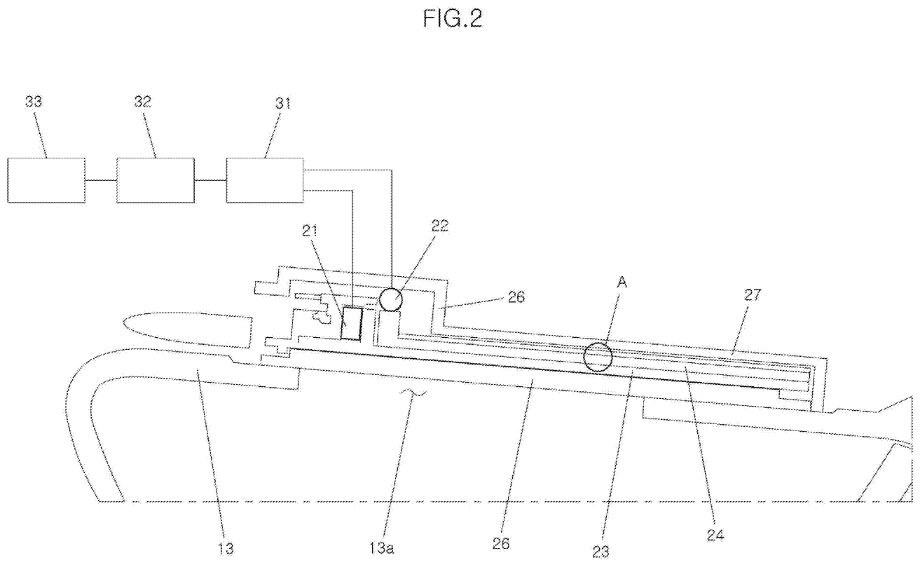

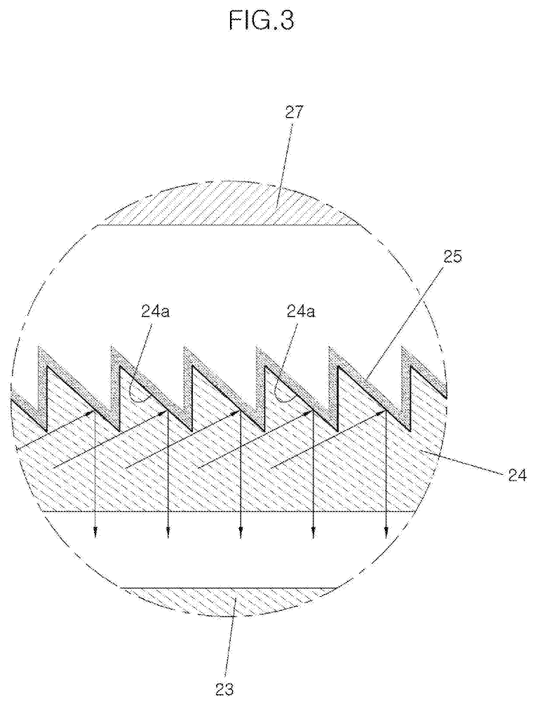

As in FIG. 1, FIG. 2, and FIG. 3, an example in which the sterilizing lamp 22 is mounted at a door inside handle 12, which is mounted at a door trim 11 of a door 10 to be operated by the passenger, will be described in detail. Since the door inside handle 12 frequently contacts with the hands of the passenger, bacteria tend to be generated. Therefore, it is possible to allowing the sterilizing lamp 22 to illuminate the door inside handle 12, sterilizing it.

An opening 13a is formed in a handle housing 13 of the door inside handle 12 so that the light irradiated from the sterilizing lamp 22 can pass through.

In the interior material, that is, the handle housing 13, an external cover 26 is mounted to cover the opening 13a, and an internal cover 27 fastened to the external cover 26 to form a space therein is mounted. The external cover 26 is made of a transparent material so that light can pass through.

The sterilizing lamp 22 is mounted in the space formed by the external cover 26 and the internal cover 27, inside the handle housing 13.

The sterilizing lamp 22 is a lamp for irradiating sterilizable light of wavelength, and at least one sterilizing lamp 22 is mounted.

The sterilizing lamp 22 is a UV Light Emitting Diode (UV LED) that irradiates Ultraviolet rays (UV), particularly, a UV-C LED that irradiates the UV having a wavelength of 200 nm to 280 nm excellent for sterilization (UV-C). The UV-C LED is smaller than a mercury lamp conventionally used as ultraviolet lamps, and does not include mercury, being eco-friendly, safe, and also durable. Therefore, when the sterilizing lamp 22 is turned on, a region where the light (the ultraviolet rays) irradiated by the sterilizing lamp 22 reaches is sterilized.

The inside of the handle housing 13 may be provided with another lamp for illuminating the interior of the vehicle in addition to the sterilizing lamp 22. For example, provided may be a general lamp for brightly illuminating the interior of the vehicle to identify objects, or a mood lamp 21 for mood illumination, which does not brighten a wide area but softly illuminates the indoor, enhancing the interior effect.

The general lamp and the mood lamp 21 are illuminated in a state where the passenger has gotten on the vehicle, but since the sterilizing lamp 22 is irradiated with ultraviolet rays, the lamp is preferably illuminated in a state where the passenger has not gotten on.

The mood lamp 21 is mounted adjacent to the external cover 26 to illuminate the indoor through a guide panel 23 for disperses the light to be uniformly illuminated. The light irradiated from the mood lamp 21 is dispersed while passing through the guide panel 23 to exert an effect as a surface light emission, implementing the mood illumination effect.

Meanwhile, a diffusion panel 24 for diffusing the ultraviolet rays irradiated from the sterilizing lamp 22 is mounted so that the ultraviolet rays irradiated from the sterilizing lamp 22 can also be uniformly illuminated without illuminating by concentrating any one spot. When the sterilizing lamp 22 illuminates one side surface of the diffusion panel 24, the ultraviolet rays are diffused and illuminated inside the diffusion panel 24.

However, only any one of the guide panel 23 and the diffusion panel 24 can also be mounted if necessary to diffuse the light irradiated from the sterilizing lamp 22 and the mood lamp 21, respectively.

Meanwhile, the diffusion panel 24 is formed with a reflective surface 24a so that the light irradiated from the sterilizing lamp 22 may be efficiently irradiated to an illumination area. The reflective surface 24a is formed on the surface opposite to the surface facing the interior of the vehicle (i.e., the surface not facing the interior of the vehicle). The reflective surface 24a reflects the light irradiated from the sterilizing lamp 22 into the interior of the vehicle. The reflective surface 24a formed in a form of an inclined surface is formed repeatedly on the diffusion panel 24.

Meanwhile, since the ultraviolet rays cannot be sufficiently reflected by only the reflective surface 24a, chromium (Cr) is applied to the reflective surface 24a to form a reflective layer 25. The reflective layer 25 is formed on the reflective surface 24a, and the ultraviolet rays irradiated from the sterilizing lamp 22 are all reflected and more ultraviolet rays are irradiated to the interior of the vehicle, increasing the sterilization effect.

The sterilizing lamp 22 detects whether the passenger has gotten on so that the passenger is not exposed to ultraviolet rays, and the sterilizing lamp 22 is turned on only when the passenger does not get on (e.g., immediately after the passenger gets off). Since the ultraviolet rays are known to be harmful, the sterilizing lamp 22 may be turned on only when the passenger does not get on.

For the present purpose, the sterilizable lamp assembly of the vehicle may include a passenger detecting sensor 33 for detecting whether the passenger has gotten on the vehicle, a controller 31 for applying power to the sterilizing lamp 22, and an Integrated Central control Unit (ICU) 32 for receiving an output signal of the passenger detecting sensor 33 to transfer whether the passenger has gotten on to the controller 33.

The passenger detecting sensor 33 is mounted inside the vehicle to determine whether the passenger has gotten on the vehicle. For example, a sensor is mounted to determine whether an airbag operates at the time of collision by determining the load acting on the seat, or to detect whether the passenger has gotten on to prevent the door from being open when an object approaches the rear side of the vehicle, and this may be used.

The ICU 32 determines whether the passenger has gotten on by the output signal of the passenger detecting sensor 33, and outputs a turning-on signal of the sterilizing lamp 22 to the controller 31 when the passenger does not get on. Since the ICU 32 is an Electronic Control Unit (ECU) that integrally controls the vehicle body length, the ICU 32 receives a signal of the passenger detecting sensor 33, and controls the controller 31 for turning on or off the sterilizing lamp 22. The ICU 32 receives the output signal from the passenger detecting sensor 33 immediately after the passenger gets off to determine whether the passenger has gotten on the vehicle.

The controller 31 applies power to the sterilizing lamp 22 according to an instruction of the ICU 32 to turn on the sterilizing lamp 22 or turns off the power applied to the sterilizing lamp 22 to turn off the sterilizing lamp 22.

According to the present configuration, when it is determined that the passenger does not get on the vehicle, the sterilizing lamp 22 is turned on to sterilize the interior of the vehicle, that is, the door inside handle 12.

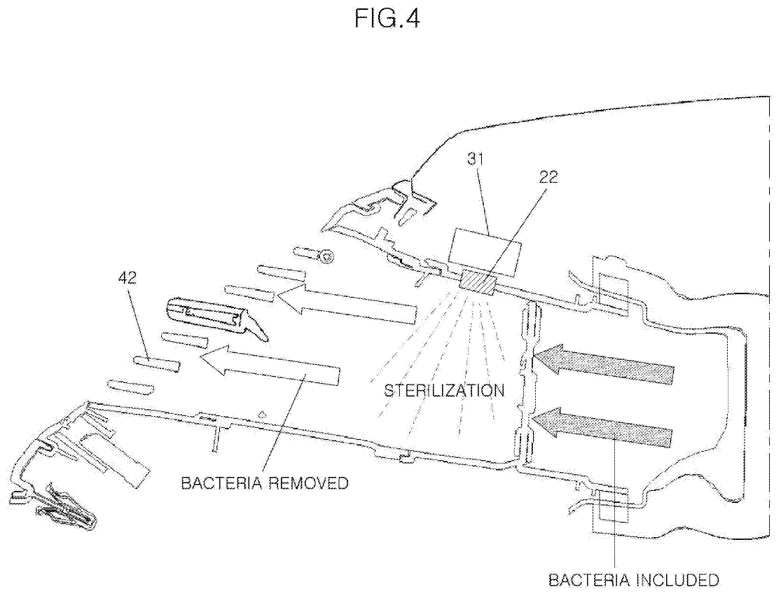

FIG. 4 is a diagram illustrating a configuration in which the sterilizing lamp 22 has been mounted in an air blowing duct 41. The air blowing duct 41 is connected and mounted to the indoor material of the vehicle such as a dashboard, and becomes a passage that provides the air conditioned by heating and cooling to the interior of the vehicle. At the end portion of the air blowing duct 41, a grill 42 for adjusting whether to blow the air and the air blowing direction is provided.

In the air blowing duct 41, bacteria increase inside the air blowing duct 41, an evaporator, etc., and the bacteria flow into the indoor together with the wind at the air blowing.

To prevent this, it is possible to install the sterilizing lamp 22 at the air blowing duct 41, sterilizing bacteria that are present in the air passing through the air blowing duct 41.

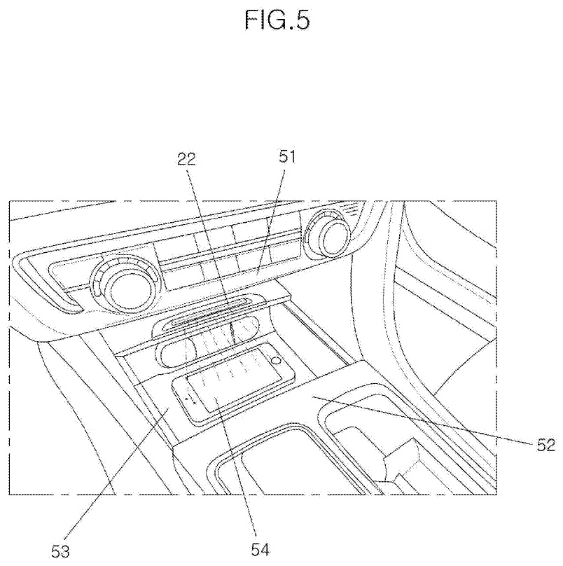

FIG. 5 is a diagram illustrating an example in which the sterilizing lamp 22 is mounted at a crash pad 51 to sterilize and illuminate a mobile phone holder 53.

In the interior of the vehicle, the mobile phone holder 53 for holding a mobile phone 54 of the passenger or allowing wireless charging while holding is formed at a console 52, etc. The mobile phone 54 reproduces various germs as the passenger always carries and frequently operates it by hand. Therefore, the sterilizing lamp 22 is mounted at the crash pad 51, which is one of the indoor materials of the vehicle, and illuminates the mobile phone holder 53.

When the mobile phone holder 53 has a wireless charging function, the mobile phone holder 53 detects a state where the mobile phone 54 has been held, and then starts charging. Therefore, in the example in which the sterilizing lamp 22 has been mounted to illuminate the mobile phone holder 53, the sterilizing lamp 22 is turned on by interlocking with the start of the wireless charging.

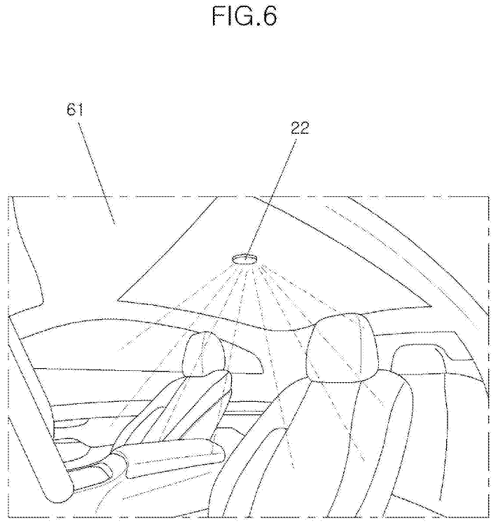

FIG. 6 is a diagram illustrating a configuration in which the sterilizing lamp 22 has been mounted in a headlining 61 of the interior of the vehicle.

The sterilizing lamp 22 is mounted at the headlining 61 and irradiates the ultraviolet rays to the interior of the vehicle when the sterilizing lamp 22 is turned on, sterilizing the interior of the vehicle.

The headlining 61 is provided with a general lamp for illuminating the interior of the vehicle when the door is open or by an operation of the passenger, or a mood lamp for mood illumination, and furthermore, the sterilizing lamp 22 may be mounted, sterilizing the interior of the vehicle.

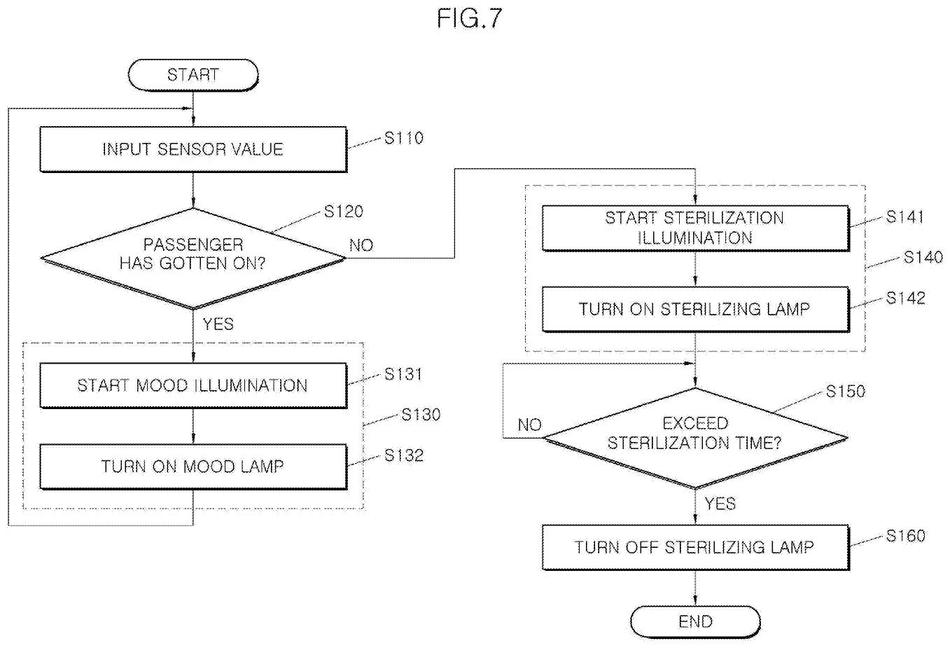

Meanwhile, FIG. 7 is a diagram illustrating a sterilization method using the sterilizable lamp assembly of the vehicle according to an exemplary embodiment of the present invention.

The sterilization method according to an exemplary embodiment of the present invention is conducted by use of the above-described sterilizable lamp assembly of the vehicle.

The sensor value input step S110 is a step in which the ICU 32 receives the sensor value from the passenger detecting sensor 33 mounted in the vehicle. Since the irradiation of ultraviolet rays for sterilization may be conducted while the passenger is not boarding the ICU 32, the ICU 32 receives the sensor value from the passenger detecting sensor 33 to determine whether or not the passenger is on board.

Inputting a sensor value S110 is to input a sensor value from the passenger detecting sensor 33 mounted in the vehicle to the ICU 32. Since the irradiation of the ultraviolet rays for sterilization may be performed in a state where the passenger has not gotten on the vehicle, the ICU 32 receives the sensor value from the passenger detecting sensor 33 to determine whether the passenger has gotten on the vehicle.

Determining whether the passenger has gotten on S120 determines whether the passenger has gotten on the vehicle. The ICU 32 determines whether the passenger has gotten based on the value input from the passenger detecting sensor 33. Any one of illuminating mood S130 and sterilizing S140, which will be described later, may be selectively performed according to whether the passenger has gotten on the vehicle.

For example, the determining whether the passenger has gotten on S120 may be performed immediately after the passenger gets off.

The illuminating the mood S130 mood-illuminates the interior of the vehicle with the mood lamp 21 mounted adjacent to the sterilizing lamp 22, when it is determined that the passenger has gotten on the vehicle. When the passenger is present, the sterilization through the ultraviolet rays may be harmful, such that the mood lamp 21 is turned on in a state where the sterilizing lamp 22 has been turned off to perform the mood illumination for interior when the passenger gets on.

The illuminating the mood S130 may include starting mood illumination that outputs the turning-on signal of the mood lamp 21 to the controller 31 when it is determined that the passenger has gotten on the vehicle by the ICU 32 S131, and turning on the mood lamp that turns on the mood lamp 21 by applying power to the mood lamp 21 when receiving the turning-on signal of the mood lamp 21 from the ICU 32 by the controller 31 S132.

After the illuminating the mood S130 has been performed, it is returned to the first step, that is, the inputting the sensor value S110 or the determining whether the passenger has gotten on S120 to perform repeatedly while the passenger gets on.

In the determining whether the passenger has gotten S120, when it is determined that the passenger has not gotten on the vehicle, the sterilizing S140 is performed to sterilize the interior of the vehicle by turning on the sterilizing lamp 22.

The sterilizing S140 may include starting sterilizing illumination that outputs the turning-on signal of the sterilizing lamp 22 to the controller 31 when it is determined that the passenger has not gotten on by the ICU 32 S141, and turning on the sterilizing lamp that applies power to the sterilizing lamp 22 to turn on the sterilizing lamp 22 when receiving the turning-on signal of the sterilizing lamp 22 from the ICU 32 by the controller 31.

The starting the sterilizing illumination S141 outputs the turning-on signal of the sterilizing lamp 22 to the controller 31 for applying power to the sterilizing lamp 22 to turn on the sterilizing lamp 22, after the ICU 32 determines that the passenger has gotten on the interior of the vehicle.

The turning on the sterilizing lamp S142 turns on the sterilizing lamp 22 so that the ultraviolet rays are irradiated to a portion requiring sterilization. When the controller 31 applies power to the sterilizing lamp 22, the sterilizing lamp 22 is turned on to irradiate ultraviolet rays to the interior of the vehicle. When the ultraviolet rays are irradiated, the bacteria exposed to the ultraviolet rays are sterilized, enhancing the cleanliness of the interior of the vehicle.

It is difficult to maintain the cleanliness of the vehicle when a plurality of passengers use a single vehicle (e.g., a vehicle sharing service), but it is possible to enhance the cleanliness only by sterilizing the interior of the vehicle by use of the sterilizing lamp 22.

Determining whether to exceed a sterilization time S150 determines whether the time in which the sterilizing lamp 22 has been turned on has exceeded a predetermined sterilization time so that the sterilizing lamp 22 is turned off. The ICU 32 measures the time during which the sterilizing lamp 22 has been turned on to determine whether the turning-on time of the sterilizing lamp 22 has exceeded a predetermined sterilization time, that is, from the time in which the sterilizing lamp 22 has been turned on to the time in which the sterilizing lamp 22 may be turned off.

The sterilizing lamp 22 irradiates ultraviolet rays so that the sterilizing lamp 22 is turned on only for a predetermined time period.

The sterilization using the sterilizing lamp 22 is sufficient to turn on the sterilizing lamp 22 for the predetermined time period, and even when ultraviolet rays are irradiated more than it, there is substantially no additional sterilization effect, turning on it for the predetermined time period, that is, only for the predetermined sterilization time.

However, there is the sterilization effect only when the sterilizing lamp 22 is turned on at least during the predetermined sterilization time, such that when the turning-on time of the sterilizing lamp 22 is less than the predetermined sterilization time, the sterilizing lamp 22 is continuously turned on for that time.

In turning off the sterilizing lamp S160, the controller blocks the power applied to the sterilizing lamp 22 so that the sterilizing lamp 22 is turned off, when it is determined that the sterilizing lamp 22 has been turned off for the predetermined time period.

For convenience in explanation and accurate definition in the appended claims, the terms “upper”, “lower”, “inner”, “outer”, “up”, “down”, “upwards”, “downwards”, “front”, “rear”, “back”, “inside”, “outside”, “inwardly”, “outwardly”, “internal”, “external”, “inner”, “outer”, “forwards”, and “backwards” are used to describe features of the exemplary embodiments with reference to the positions of such features as displayed in the figures. It will be further understood that the term “connect” or its derivatives refer both to direct and indirect connection.

The foregoing descriptions of specific exemplary embodiments of the present invention have been presented for purposes of illustration and description. They are not intended to be exhaustive or to limit the present invention to the precise forms disclosed, and obviously many modifications and variations are possible in light of the above teachings. The exemplary embodiments were chosen and described to explain certain principles of the present invention and their practical application, to enable others skilled in the art to make and utilize various exemplary embodiments of the present invention, as well as various alternatives and modifications thereof. It is intended that the scope of the present invention be defined by the Claims appended hereto and their equivalents.

What is claimed is:

1. A sterilizable lamp assembly of a vehicle, the assembly comprising:
at least one sterilizing lamp mounted in a vehicle and of irradiating sterilizable light of wavelength to an interior of the vehicle,
wherein the at least one sterilizing lamp is configured to emit light when a passenger is determined to not get on the interior of the vehicle.

2. The sterilizable lamp assembly of the vehicle of claim 1,
wherein the at least one sterilizing lamp irradiates ultraviolet rays of 200 nm to 280 nm.

3. The sterilizable lamp assembly of the vehicle of claim 1,
wherein the at least one sterilizing lamp is a UV-C LED.

4. The sterilizable lamp assembly of the vehicle of claim 1,
wherein an opening through which the light irradiated from the at least one sterilizing lamp passes is formed at an interior material of the vehicle, and
wherein an inside of the interior material includes a diffusion panel for dispersing the light irradiated from the at least one sterilizing lamp.

5. The sterilizable lamp assembly of the vehicle of claim 4,
wherein the diffusion panel has a reflective surface formed to be inclined to reflect the light irradiated from the at least one sterilizing lamp to the opening, the reflective surface being repeatedly formed on the diffusion panel in a predetermined length of the diffusion panel.

6. The sterilizable lamp assembly of the vehicle of claim 5,
wherein a rear surface of the reflective surface is formed with a reflective layer on which chrome is coated.

7. The sterilizable lamp assembly of the vehicle of claim 1, further including
a controller configured of turning on the at least one sterilizing lamp; and
an Integrated Central control Unit (ICU) connected to the controller and of outputting a turning-on signal of the at least one sterilizing lamp to the controller, when the passenger is detected to not have gotten on the vehicle by a passenger detecting sensor of detecting whether the passenger has gotten on the interior of the vehicle.

8. The sterilizable lamp assembly of the vehicle of claim 1,
wherein the at least one sterilizing lamp is mounted inside a handle housing of a door inside handle to illuminate the door inside handle.

9. The sterilizable lamp assembly of the vehicle of claim 1,
wherein the at least one sterilizing lamp is mounted inside an air blowing duct configured for blowing air to the interior of the vehicle.

10. The sterilizable lamp assembly of the vehicle of claim 1,
wherein the at least one sterilizing lamp is mounted at a crash pad of the vehicle to illuminate a mobile phone holder in which a mobile phone is configured to be held for wireless charging.

11. The sterilizable lamp assembly of the vehicle of claim 1,
wherein the at least one sterilizing lamp is mounted at a headlining of the interior of the vehicle to illuminate the interior of the vehicle.

12. The sterilizable lamp assembly of the vehicle of claim 1,
wherein a mood lamp for mood-illuminating the interior of the vehicle is mounted at a position adjacent to the at least one sterilizing lamp.

13. The sterilizable lamp assembly of the vehicle of claim 1, further including:
an opening formed in a handle housing of a door inside handle so that the light irradiated from the at least one sterilizing lamp passes through the opening;
an external cover mounted to cover the opening, wherein the external cover is made of a transparent material so that light passes through the opening; and
an internal cover fastened to the external cover to form a space between the internal cover and the external cover,
wherein the at least one sterilizing lamp is mounted in the space formed by the external cover and the internal cover, inside the handle housing.

14. A sterilization method using a sterilizable lamp assembly of a vehicle, the method comprising:
in order to sterilize an interior of the vehicle by use of at least one sterilizing lamp mounted in the interior of the vehicle to irradiate sterilizable light of wavelength to the interior of the vehicle,
determining whether a passenger has gotten on the interior of the vehicle; and
sterilizing the interior of the vehicle by turning on the at least one sterilizing lamp, when the passenger is determined, by a controller, to have not gotten on the vehicle.

15. The sterilization method using the sterilizable lamp assembly of the vehicle of claim 14, further including
receiving, by an Integrated Central control Unit (ICU), a sensor value that inputs a sensor value from a passenger detecting sensor mounted in the vehicle, before the determining whether the passenger has gotten on the vehicle.

16. The sterilization method using the sterilizable lamp assembly of the vehicle of claim 15,
wherein the sterilizing the interior of the vehicle includes:
outputting, by the ICU, a turning-on signal of the at least one sterilizing lamp to the controller, when the passenger is determined to not have gotten on the vehicle; and
turning on, by the controller, the at least one sterilizing lamp by applying power to the at least one sterilizing lamp, when the controller receives the turning-on signal of the at least one sterilizing lamp from the ICU.

17. The sterilization method using the sterilizable lamp assembly of the vehicle of claim 14, further including, after the sterilizing the interior of the vehicle has been performed,
determining whether a time in which the at least one sterilizing lamp has been turned on has exceeded a predetermined sterilization time; and
turning off the at least one sterilizing lamp when the time in which the at least one sterilizing lamp has been turned on is determined to have exceeded the predetermined sterilization time.

18. The sterilization method using the sterilizable lamp assembly of the vehicle of claim 17,
wherein when the time in which the at least one sterilizing lamp has been turned on is determined to have not exceeded the predetermined sterilization time,
repeatedly performing the determining whether the time in which the at least one sterilizing lamp has been turned on has exceeded the predetermined sterilization time so as to maintain a state where the at least one sterilizing lamp has been turned on for the predetermined sterilization time.

19. The sterilization method using the sterilizable lamp assembly of the vehicle of claim 15,
wherein when the passenger is determined to have gotten on the vehicle,
performing an illuminating mode in which the interior of the vehicle is illuminated with a mood lamp mounted adjacent to the at least one sterilizing lamp.

20. The sterilization method using the sterilizable lamp assembly of the vehicle of claim 19,
wherein the illuminating mode includes:
outputting, by the ICU, a turning-on signal of the mood lamp to the controller when the passenger is determined to have gotten on the vehicle; and
turning on the mood lamp by applying power to the mood lamp, when the controller receives the turning-on signal of the mood lamp from the ICU.
